(19) United States
(12) Patent Application Publication
(10) Pub. No.: US 2002/0103425 A1
(43) Pub. Date: Aug. 1, 2002

(54) self-contained monitoring device particularly useful for monitoring physiological conditions

(76) Inventor: James R. Mault, Evergreen, CO (US)

Correspondence Address: GIFFORD, KRASS, GROH, SPRINKLE,, ANDERSON & CITKOWSKI, P.C., Suite 400, 280 N. Old Woodward, Birmingham, MI, 48009, US

(21) Appl. No.: 09/963,699

(22) Filed: Sep. 26, 2001

Related U.S. Application Data
(60) Provisional application No. 60/235,739, filed on Sep. 27, 2000.

(51) Int. Cl. A61B 5/04

(52) U.S. Cl. 600/373

(57) ABSTRACT

A monitoring device to be applied to an object for monitoring a condition of the object includes a housing configured and dimensioned for application to the object; a sensor within the housing for sensing the condition to be monitored and for producing an electrical output corresponding to the sensed condition; and an electrical power generator within the housing for receiving energy from a source externally of the housing and for generating, from the received energy, electrical power for energizing the sensor. The monitoring device is particularly useful for monitoring a physiological condition of a living subject, e.g. by implanting the monitoring device, swallowing it or attaching it to the external skin of the subject.

### RELATED APPLICATION

This application claims priority of U.S. Provisional Patent Application No. 60/235,739 filed Sep. 27, 2000 and is incorporated herein by reference.

### FIELD AND BACKGROUND OF THE INVENTION

The present invention relates to a self-contained monitoring device for monitoring various conditions of an object. The invention is particularly useful for application to a living object for monitoring various physiological conditions of the living object, and the invention is therefore described below with respect to such applications.

There is great interest in monitoring various physiological conditions of persons by the use of skin-mounted, under-skin mounted, or implanted sensors. Since such sensors should be made as small and unobtrusive as possible, this limits the size of the battery power supply. Moreover, such sensors are difficult to remove for battery-changing or battery-charging.

### OBJECTS AND BRIEF SUMMARY OF THE INVENTION

A broad object of the present invention is to provide a monitoring device, having advantages in the above respects, applicable to an object for monitoring a condition of the object. A more particular object of the invention is to provide a monitoring device particularly useful for application to a living object such as a person or experimental animals for monitoring a physiological condition of the living object.

According to a broad aspect of the present invention, there is provided a monitoring device for application to an object for monitoring a condition of the object, comprising: a housing configured and dimensioned for application to the object; a sensor within the housing, for sensing the condition to be monitored and for producing an electrical output corresponding to the sensed condition; and an electrical power generator within the housing for receiving energy from a source externally of the housing and for generating, from the received energy, electrical power for energizing the sensor.

In the preferred embodiments of the invention described below, the object is a living object, and the sensor is one sensing a physiological condition of the living object.

Embodiments are described below wherein the housing is configured and dimensioned for implantation within the living object for swallowing, or for attachment to the outer skin of the living object.

In some described preferred embodiments, the electrical power generator within the housing generates electrical power from electromagnetic energy, such as radio frequency (RF) energy, a light energy, or infrared (IR) energy, applied from a source externally of the housing. In other described embodiments, the electrical power generator within the housing generates electrical power from thermal energy, mechanical energy, or ultrasonic energy, applied from a source externally of the housing.

Embodiments are also described wherein the housing further includes a memory device for storing the electrical output from the sensor or an antenna for transmitting, to a receiver externally of the housing, the electrical output from the sensor within the housing. A further embodiment is described wherein the housing also includes a power storage device, such as a capacitor or a chargeable battery, for storing power generated by the electrical power generator.

Embodiments are described below wherein the housing is configured and dimensioned: for implantation in a blood vessel of a living subject, and the sensor senses the glucose level of the blood within the blood vessel; for swallowing by a living subject, and the sensor senses a condition of the digestive tract of the living subject, such as the pH level of the stomach; and for attachment to a blood vessel, and the sensor senses blood flow velocity through the blood vessel. A still further embodiment is described wherein the monitoring device includes a transponder which, when triggered by an external device, transmits the electrical output of the sensor to an external receiver.

As will be described more particularly below, a monitoring device constructed in accordance with the foregoing features may include a sensor which is powered by an external source via its internal power generator, and therefore does not require removal of the monitoring device for battery-changing or battery-charging. This makes the monitoring device particularly useful for application to a living subject, e.g. by implantation, to monitor a physiological condition of the subject. The output of the sensor, which monitors the physiological condition, may be transmitted in a wireless manner to a receiver externally of the subject, or may be recorded in a memory within the monitoring device for removal when this information is to be retrieved.

Further features and advantages of the invention will be apparent from the description below.

### BRIEF DESCRIPTION OF THE DRAWINGS

The invention is herein described, by way of example only, with reference to the accompanying drawings, wherein:

FIG. 1 diagrammatically illustrates one application of the invention for mounting a monitoring device to the external skin of a living subject;

FIG. 2 diagrammatically illustrates another application of the invention in which the monitoring device is mounted under the skin (subcutaneously);

FIG. 3 diagrammatically illustrates an example of an implanted monitoring device mounted on the outside surface of a blood vessel (endoluminal), e.g. for measuring blood flow through the blood vessel;

FIG. 4 diagrammatically illustrates the monitoring device mounted within a blood vessel (intraluminally), for blood analysis, (e.g. to detect the glucose level), cardiac output, or for another like purpose;

FIGS. 5A and 5B illustrate the monitoring device constructed in the shape of a pill or tablet for swallowing by the subject;

FIG. 6 illustrates a monitoring device which is implanted and equipped with a transponder for transmitting the information as to the physiological condition sensed by the sensor in the monitoring device;

FIG. 7 diagrammatically illustrates an implantable monitoring device equipped with an antenna for the wireless transmission of the sensed information, and also with a power storage device for storing power within the monitoring device as applied from an external power source;

FIG. 8 illustrates an implantable monitoring device including a piezoelectric crystal for powering the sensor within the device by mechanical energy supplied from a source externally of the monitoring device; and

FIG. 9 diagrammatically illustrates an implantable monitoring device including a photocell for utilizing light energy externally of the monitoring device for powering the sensor within the monitoring device.

### DESCRIPTION OF THE PREFERRED EMBODIMENTS

While the invention may be used for constructing monitoring devices for application to various types of objects for monitoring a condition of the object, the invention is particularly useful for application to living subjects, as by skin mounting, under-skin mounting, implantation, or swallowing, for monitoring a physiological condition of the subject. The preferred embodiments of the invention described below are therefore constructed for such applications.

Generally speaking, such monitoring devices include a housing configured and dimensioned for application to the object, a condition of which is to be monitored; a sensor within the housing for sensing the condition to be monitored and for producing an electrical output corresponding thereto; and an electrical power generator within the housing for receiving energy from a source externally of the housing and for generating, from the received energy, electrical power for energizing the sensor.

The drawings illustrate various types of such monitoring devices for various applications.

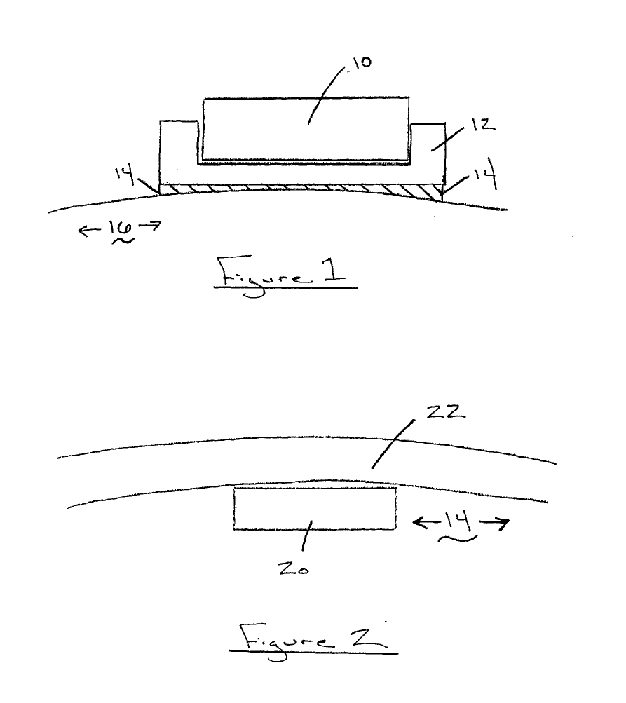

FIG. 1 shows a physiological monitoring device 10 mounted on the skin of a person 16. Device 10 is carried by a clip 12 held on the skin of the person 16 by an adhesive layer 14. Monitoring device 10 may be used to record skin temperature or conductivity, to measure physical activity, to emit and detect ultrasound radiation, etc. The monitoring device is powered in a wireless manner as described below.

In one embodiment, the monitor may tale the physical form of a computer and include a memory, such as a memory card, and an electrical interface so that it may be removed from the clip and plugged into, for example, a PDA or other portable electronic device. The advantages of wireless power in this case include reduced size. The monitoring device may also contain a capacitor or battery which is recharged using wireless methods.

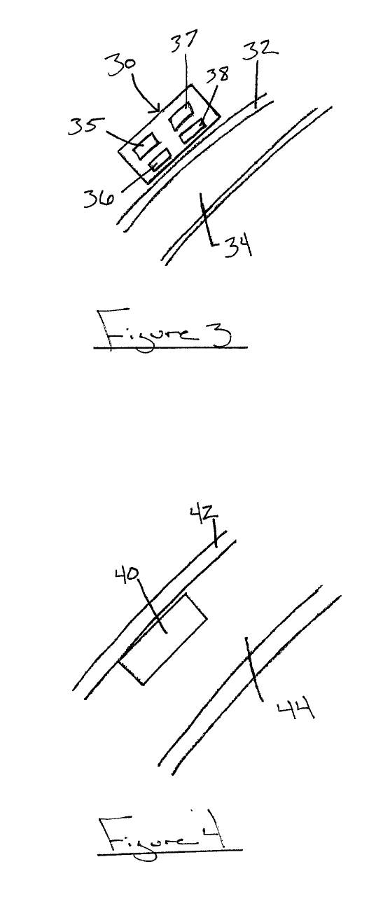

FIG. 2 shows an example of an implanted monitoring device 20. In this case, the monitoring device is mounted under the skin 22 of the subject 24, i.e., subcutaneously. The monitor housing may contain blood sensing and blood analysis sensors, such as glucose sensors and may use wireless transmission for energizing the sensor therein, as well as for transmitting its electrical output, as discussed more particularly below.

FIG. 3 shows another example of an implanted monitoring device 30 mounted on the outer wall surface 32 of a blood vessel 34. This configuration is known as an endoluminal monitor. Preferably, the monitoring device 30 contains at least one ultrasonic transducer sensor 35 and control circuitry 36 so as to measure blood flow through the vessel 32. The monitor may also have blood analysis capabilities. The sensor 35 and control circuitry 36 are both powered by a power generator 37 which receives its power from an external device in a wireless manner, as to be described more particularly below. Monitoring device 30 further includes an output device 38, such as a memory or transponder, for outputting the output of the sensor 35, as also described below.

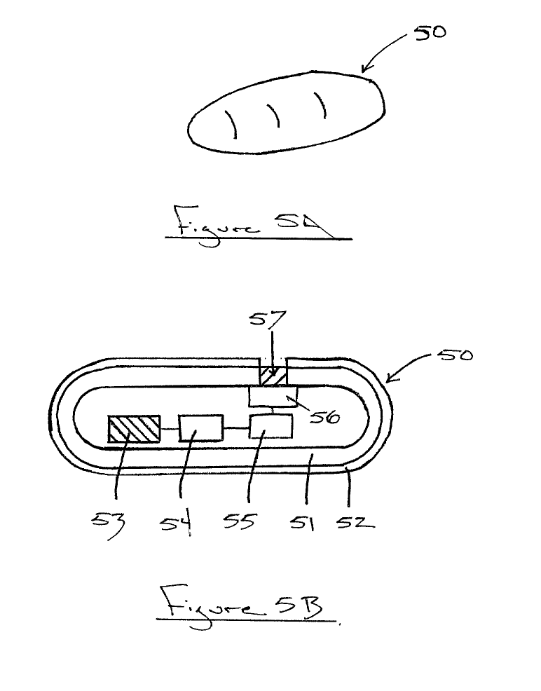

FIG. 4 shows an intraluminal-monitoring device 40 mounted on the inner surface of the wall 42 of a blood vessel 44. The housing of the monitor preferably contains a micromachined ultrasonic transducer (not shown), to measure blood flow through the vessel. A blood analysis sensor may also be mounted within the monitor housing. Cardiac output may be monitored using endoluminal or intraluminal sensors.

In the above-described embodiments, as well as in others to be described below, the physiological condition monitors described may receive power from electromagnetic radiation, such as:

(a) electromagnetic radiation at the frequency of mains electricity distribution (60 Hz in the U.S.);

(b) wireless transmissions, e.g. radio frequency (RF), commercial radio, cell phones, etc.

(c) radiation emitted by another device carried by the person, such as a personal digital assistant (PDA), watch, cell phone, organizer, pager, or other electronic device;

(d) ambient light, e.g. sunlight, artificial light, ambient IR (infrared) radiation; and

(e) other IR radiation such as thermal radiation.

For example, the IR emission of a personal digital assistant (PDA) may irradiate a photocell within the housing of a physiological condition monitor. The housing material or a section thereof may be chosen to be light-transmissive of the radiation used to power the monitor and/or its sensor.

In other embodiments of the present invention, the monitors may derive electrical power using one of the following methods:

(a) from a piezoelectric crystal under the effects of movement such as physical activity, or under irradiation by acoustic waves such as ambient sound or ultrasound radiation;

(b) from the thermoelectric effect, for example using temperature gradients near the skin, or localized heating effects such as using an IR beam; and

(c) from thermal effects, e.g. the effect of expansion on a piezoelectric crystal.

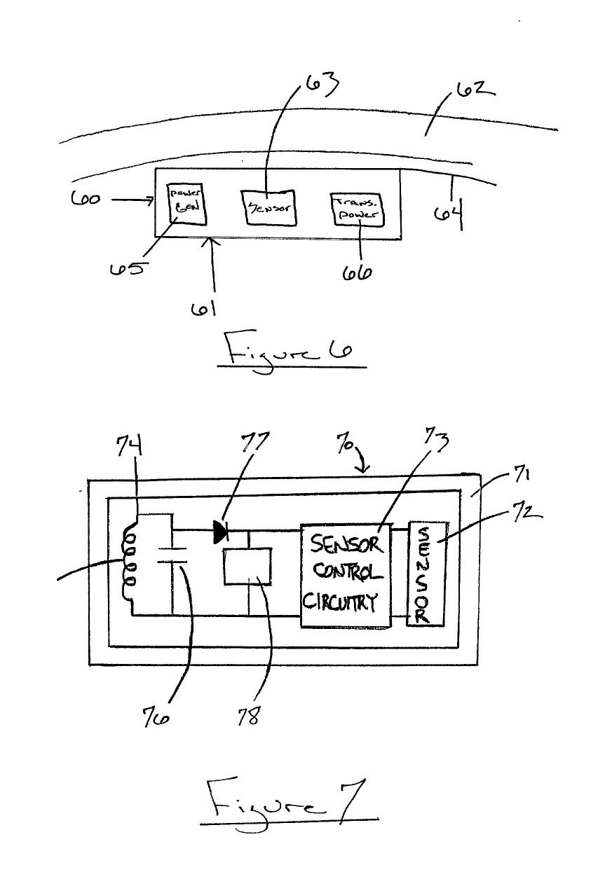

FIGS. 5A and 5B show a physiological monitoring device 50 in the shape of a pill or tablet which can be swallowed. FIG. 5A shows the outer appearance of the monitoring device 50; FIG. 5B diagrammatically shows its contents.

Monitoring 50 includes a rigid housing 51 to give strength to the structure; an outer layer 52 to reduce irritation of the intestinal tract; a wireless power circuit 53 which converts wireless energy to electrical power; control and memory circuitry 54; sensor control circuitry 55; a pH sensor 56; and a permeable membrane 57 in a section of the housing to allow fluids to contact the pH sensor 42. Monitor 50 is swallowed by a person and passes through the digestive tract of the person. For example, it can be used to monitor the pH of the stomach and/or intestines. The sensed values may be stored in a memory within the housing of the sensor, or transmitted by wireless means to a device outside of the person's body.

In other embodiments, the swallowed monitor may contain sensors for diagnosing and/or treating disease or infection.

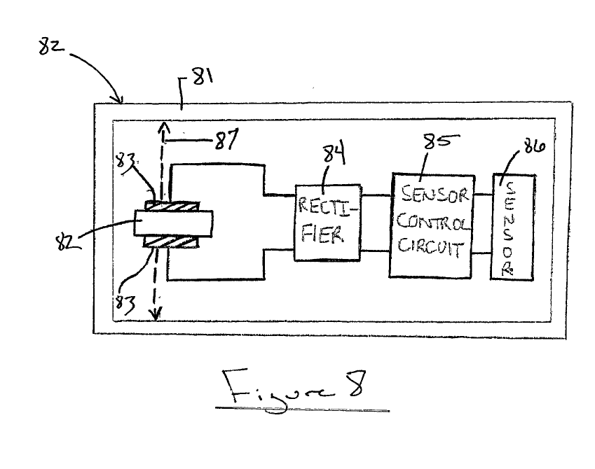

FIG. 6 shows a monitoring device, generally designated 60, including a housing 61 to be implanted within the subject's body, such as under the subject's skin 62. Housing 61 includes a sensor 63 for sensing a predetermined physiological condition of the subject. Housing 61 also includes an external antenna 64 which may be in the form of a rigid wire or flexible conductor implanted under the skin. An antenna wire may also be wound around the housing of the monitor, or may be contained within the housing.

The monitoring device 60 illustrated in FIG. 6 further includes a power generator, generally designated 65, for utilizing power supplied from an external source to energize the sensor 63. For example, power generator 65 may be an electrical tuned circuit, such as described below with respect to FIG. 7.

In the embodiment illustrated in FIG. 6, the monitoring device 60 further includes a transponder, generally designated 66, which is effective, when triggered by an external device, to transmit the output of the sensor 63 to an external receiving device (not shown). Such a transponder can be included in any of the other discussed embodiments.

FIG. 7 shows a schematic of a monitoring device, generally designated 70, including a housing 71 constructed and dimensioned so as to be implantable within the body of a subject. Housing 71 houses a sensor 72 for sensing a physiological condition of a subject, and sensor control circuitry 73 for controlling sensor 72. Monitoring device 70 further includes an antenna 74, an inductor 75, a capacitor 76, a diode rectifier 77, and a voltage storage device 78. The antenna 74 may be extended around the housing 71, or within the housing 71. The inductor 74 and capacitor 76 are chosen so as to tune in a strong AM (or FM) radio station. The voltage provided by the detected and rectified AM signal is used to power the physiological condition sensor 72.

The storage device 78 stores the rectified voltage generated by the circuit elements 75, 76, 77, so as to provide uninterrupted power to the sensor 72 and its control circuitry 73. Storage device 78 may be, for example, a capacitor for storing the rectified voltage, or a battery rechargeable by the rectified voltage.

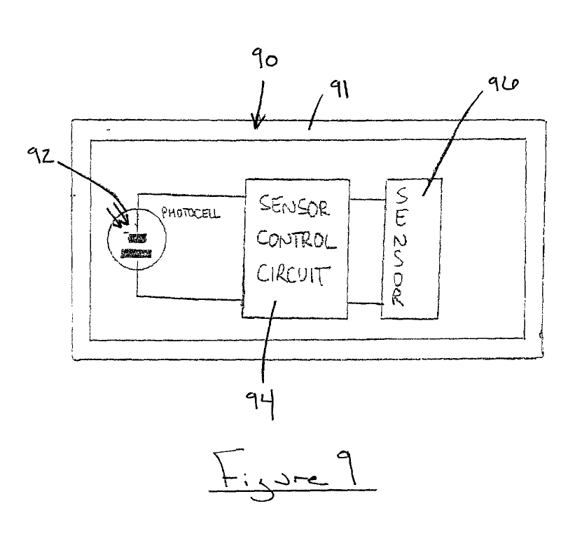

FIG. 8 shows another embodiment of implantable monitoring device 80. It includes housing 81 containing a piezoelectric crystal 82 with attached electrodes 83. Electrical output from the crystal is passed to a rectifier circuit 84, and the DC power from the rectifier is passed to sensor control circuit 85. The circuit 85 is used to power, control and store data from the sensor 86. Electrical power is derived from mechanical deformation of the crystal 82. The crystal may have a mechanical coupling to the housing 81 or a section thereof, so that deformation of the housing or section induces a voltage from the piezoelectric crystal 82. For example, the housing 81, or a part of it, may be pressure-deformable and mechanically coupled, as shown schematically at 87, to the piezoelectric crystal 82 so as to mechanically transmit forces thereto.

Crystal 82 may be deformed by various forms of physical actions, such as walking, running, jumping on the spot, posture changes, swallowing, chewing (particularly for a monitor in the mouth), respiration, cardiac activity, blood pulse, impact with the ground (if in the foot), massage, scratching the skin, speaking, physical impact, muscle activity, etc. Alternatively, the crystal 82 may provide a voltage in response to acoustic waves and vibrations, such as speech, ambient noise, or a vibrating object placed against the body. The crystal 82 may also provide a voltage in response to irradiation with ultrasonic waves.

Another ultrasonic transducer corresponding to piezoelectric crystal 82 may also be mounted on the skin and used to power a subcutaneous or embedded monitor 80 in the body. A PDA, pen, or other portable device may contain an ultrasonic transducer which when brought close to the skin powers a subcutaneous monitor. Data may also be transmitted to the PDA at this time. A PDA may prompt the person to power the monitor, e.g. by pressing on the skin, talking to the monitor, or bringing an ultrasonic transducer close to the monitor. A PDA, such as one containing the functionality of a wireless telephone, may be placed on the skin and may be caused to vibrate to power a subcutaneous monitor.

FIG. 9 shows another implantable monitoring device, generally designated 90, including a housing 91 constructed and dimensioned so as to be implantable in the body of a subject. In this case, housing 91 of the monitoring device contains a photoelectric device 92 in alignment with a light-transmissive section of the housing. Optical, IR, or UV radiation falling on the photocell may be used to generate a photocurrent which may then be used to power the sensor control circuitry 94 and sensor 96. If the voltage obtained from a single photocell is too low, particularly if IR radiation is used, a number of photocells may be used, placed in series to obtain sufficient voltage to power the sensor and sensor control circuit. An IR beam from the PDA may also be used to power the monitor, e.g. in the same manner that an IR beam is conventionally used for data transfer. Laser radiation, such as from a bar code scanner in a PDA, may also be used to power the monitor.

It will thus be seen that monitors may be constructed in accordance with the present invention to be used in a wide variety of applications in order to detect various physiological conditions existing in the subject or produced as a result of various physical activities by the subject. Such applications include blood analysis (for which subcutaneous and other body implanted monitors are advantageous), physical activity (for which skin-mounted monitors are advantageous), cardiac output studies (for which monitors near or in blood vessels are advantageous), intestinal or stomach conditions such as pH (for which swallowed monitors are advantageous), and EKG monitoring (for which skin-mounted monitors are advantageous). Other possible applications include disease monitoring, body temperature measurement, ultrasonic flow determination, and ultrasonic imaging. Other environmental, medical and non-medical conditions may be monitored. A PDA may be carried by one person and communicate with a wireless-powered monitor associated with another, as may be advantageous in doctor-patient and parent-child relationships. The monitors may communicate with other computer systems and communications networks.

While the monitoring devices described herein are particularly useful for application to living subjects, such as human beings, animals, etc., for monitoring various physiological conditions, it will be appreciated that the monitoring devices can also be used in other applications for monitoring conditions with respect to other objects, such as environmental conditions or operating conditions of equipment and machinery. The foregoing embodiments of the invention are therefore to be considered as being merely illustrative, and that many other variations, modifications and applications of the invention may be made.

What is claimed is:

1. A monitoring device for application to an object for monitoring a condition of the object, comprising:
a housing configured and dimensioned for application to the object;
a sensor within said housing, for sensing the condition to be monitored and for producing an electrical output corresponding to the sensed condition; and
an electrical power generator within said housing for receiving energy from a source externally of said housing and for generating, from said received energy, electrical power for energizing said sensor.

2. The monitoring device according to claim 1, wherein said object is a living object, and said sensor is one sensing a physiological condition of the living object.

3. The monitoring device according to claim 2, wherein said housing is configured and dimensioned for implantation within the living object.

4. The monitoring device according to claim 2, wherein said housing is configured and dimensioned for attachment to the outer skin of the living object.

5. The monitoring device according to claim 2, wherein said housing is configured and dimensioned to be swallowed by a living object, and said sensor senses a condition of the digestive tract of the living object and produces an electrical output corresponding thereto.

6. The monitoring device according to claim 2, wherein said housing is configured and dimensioned for implantation in a blood vessel of a living object, and said sensor senses the glucose level of the blood within said blood vessel.

7. The monitoring device according to claim 2, wherein said housing is configured and dimensioned for attachment to a blood vessel in a living object, and said sensor senses blood flow velocity through said blood vessel.

8. The monitoring device according to claim 1, wherein the electrical power generator within the housing generates electrical power from electromagnetic energy applied from a source externally of the housing.

9. The monitoring device according to claim 8, wherein said electromagnetic energy is radio frequency (RF) energy.

10. The monitoring device according to claim 8, wherein said electromagnetic energy is light energy.

11. The monitoring device according to claim 8, wherein said electromagnetic energy is infrared energy.

12. The monitoring device according to claim 1, wherein the electrical power generator within the housing generates electrical power from thermal energy applied from a source externally of the housing.

13. The monitoring device according to claim 1, wherein the electrical power generator within the housing generates electrical power from mechanical energy applied from a source externally of the housing.

14. The monitoring device according to claim 1, wherein the electrical power generator within the housing generates electrical power from ultrasonic energy applied from a source externally of the housing.

15. The monitoring device according to claim 1, wherein said electrical power generator within the housing includes a tuned electrical circuit for receiving electromagnetic energy applied from a source externally of the housing.

16. The monitoring device according to claim 1, wherein said electrical power generator within the housing includes a photosensitive device for receiving light energy applied from a source externally of the housing through a light-transmissive section of the housing.

17. The monitoring device according to claim 1, wherein said electrical power generator within the housing includes a piezoelectric device for receiving mechanical energy applied from a source externally of the housing.

18. The monitoring device according to claim 17, wherein said housing includes a deformable section mechanically coupled to said piezoelectric device within the housing for converting the mechanical energy applied from the source externally of the housing to electrical power for energizing said sensor.

19. The monitoring device according to claim 1, wherein said housing further includes an antenna for transmitting, to a receiver externally of the housing, the electrical output from said sensor within the housing.

20. The monitoring device according to claim 1, wherein said housing further includes a memory device for storing the electrical output from said sensor.

21. The monitoring device according to claim 1, wherein said housing further includes a power storage device for storing power generated by said electrical power generator for use in energizing said sensor.

22. The monitoring device according to claim 1, wherein said electrical power generator within said housing generates power from thermal energy within said object.

23. The monitoring device according to claim 2, wherein said housing further includes a transponder triggered by an external source for transmitting outwardly of said housing the electrical output of the sensor within the housing.
